(19) United States
(12) Patent Application Publication
(10) Pub. No.: US 2019/0336761 A1
(43) Pub. Date: Nov. 7, 2019

(54) HANDS-FREE NERVE STIMULATING BALL APPARATUS, AND A METHOD OF USING SAME

(71) Applicant: Joo Hyeong Yoo, Chester, NY (US)

(72) Inventor: Joo Hyeong Yoo, Chester, NY (US)

(21) Appl. No.: 16/392,218

(22) Filed: Apr. 23, 2019

Related U.S. Application Data
(60) Provisional application No. 62/665,409, filed on May 1, 2018.

(51) Int. Cl. A61N 1/36

(52) U.S. Cl. CPC A61N 1/36014; A61N 1/0472; A61N 1/36007

(57) ABSTRACT

The present invention relates generally to a hands-free nerve stimulating ball apparatus, and a method of using same. More particularly, the invention encompasses an apparatus, and a method, to apply pressure to a specific area on the back, and the spine. The device, and method are particularly suited for trigger point massage which can stimulate the thoracic nerve endings thus resulting in improve digestive organ function. The invention is built to be used by a human while standing, sitting, or lying on the back. More specifically, but not exclusively, the invention further relates to a device, and method for applying deep-tissue pressure on a pair of acupressure points on a body, enabling improved digestive organ function while driving or sitting or standing, and With or without the use of the hands.

### CROSS-REFERENCE TO RELATED APPLICATION

The instant patent application claims priority to and the benefit of pending U.S. Provisional Patent Application Ser. No. 62/665,409, filed on May 1, 2018, titled “Hands-Free Nerve Stimulating Ball,” the entire disclosure of which provisional application is incorporated herein by reference.

### FIELD OF THE INVENTION

The present invention relates generally to a hands-free nerve stimulating ball apparatus, and a method of using same. More particularly, the invention encompasses an apparatus, and a method, to apply pressure to a specific area on the back, and the spine. The device, and method are particularly suited for trigger point massage which can stimulate the thoracic nerve endings thus resulting in improve digestive organ function. The invention is built to be used by a human While standing, sitting, or lying on the back. More specifically, but not exclusively, the invention further relates to a device, and method for applying deep-tissue pressure on a pair of acupressure points on a body, enabling improved digestive organ function while driving or sitting or standing, and With or without the use of the hands.

### BACKGROUND INFORMATION

Body stimulation apparatus, and massaging apparatus have been used in the industry, and by the consumers, for a variety of reasons, and they come in many shapes, sizes, and other physical features, and attributes.

Digestion is a process of breaking down food by mechanical, and enzymatic action in the alimentary canal into substances that can be used by the body. For the body to get the most benefits from the food one eats, it must be digested effectively. The organs of the body work in a balanced way to ensure effective digestion. To help speed up the digestive process, it is recommended that one engages in some activities, such as, for example, walking, exercising, etc., however, due to various reasons, one may not have the time to move around, or go for a walk, or engage in some sort of exercise after, for example, a meal. Furthermore, a person may have a meal while driving a vehicle, or traveling in an airplane, or the person may immediately need to drive a vehicle, or sit in an office chair after a meal; or the person may simply just relax after a meal which tends to slow down the digestion process. Additionally, the breakdown of certain foods in the large intestine by bacteria naturally creates gas bubbles accumulation in a person's intestines, and stomach. This can cause a feeling of fullness, and discomfort in the stomach, and chest, and may slow down the digestion process, and brings discomfort. There are many remedies for relieving digestive discomfort. Burping, or belching, for example, can help release the excess gas, and speed up the digestion process. In fact, burping is one of the first thing one can do to aid in the digestion process. For burping to be effective, it needs to be initiated quickly after a meal. Some ways to induce burping may include, for example, taking an over-the-counter antacids, or drinking carbonated water, to name a few.

One effective method, not often used, to aid in digestion, is nerve stimulation. Nerve stimulation therapy not only helps initiates burping, it also contributes to improve digestive organ function. It is based on the principle of reflexology around the thoracic spine area of the vertebral column.

For the sake of clarity, it should be noted that this invention is based on reflex therapy, and not acupressure, or acupuncture. Reflexology, and acupressure, are not interchangeable, and are based on two different body energies. While acupressure/acupunctures rely on meridians, or energy pathways, within the body, reflexology is based on different medical principles. According to researchers, reflexology, also known as, zone therapy, is the application of pressure to areas, or zones, of the body, such as, the feet, the hands, areas of the back, etc. This zone therapy is based on the principle that there are small, and specific areas, of nerve endings in the hands, feet, back, etc., that directly correspond to specific muscle groups, or organs, of the body, and glands, and can trigger a reflex in those organs.

Various methods have been used to apply pressure to the pressure points on the body. For example, the feet can be massaged, either by a practitioner, a friend, or the pressure can be self-administered by the person. While the feet are more commonly used as reflex zones in reflexology, the back has a more direct connection to the thoracic spine area on the vertebral column. In reflexology, consistent touch applied to the area of the thoracic spine on the back can be more effective for many organ functions, including, digestion.

A person may become skilled in applying the zone therapy with their own hands, or the person may ask someone else, for example, a friend, or a relative, who can help administer the pressure on the person's body, however, there are many drawbacks to the available methods of administering pressure to the pressure points on a person's body. For example, another person may not always be available to administer the pressure therapy when it is needed, or, due to the position of the pressure point on the body, the user may find it uncomfortable, and ineffective, to apply the pressure themselves, such as, on the backside of the body, etc. Furthermore, if there is help available to administer the zone pressure, the location, or the environment may not be conducive for applying the pressure discreetly. For example, the user could be in an office setting with many co-workers nearby, or at a religious services, or the person maybe driving a vehicle, etc.

Therefore, there is a need for the development of a device and a method that can help facilitate pressure to the desire pressure point. There remains the need for a product or method that can serve as a tool for applying pressure to an area, such as, the back, etc. Such a product needs to be portable, and hands-free, and it must be easy to use discreetly while, for example, sitting, driving a vehicle, or lying on one's back without the use of another person. Furthermore, such a product must be simple, and affordable.

This invention improves on the deficiencies of the prior art and provides an inventive hands-free nerve stimulating ball apparatus, and a method of using same.

### PURPOSES AND SUMMARY OF THE INVENTION

The invention is a novel hands-free nerve stimulating ball apparatus, and a method of using same.

Therefore, one purpose of this invention is to provide a novel hands-free nerve stimulating ball apparatus, and a method of using same.

Another purpose of this invention is to provide a novel hands-free nerve stimulating ball where the string or strap is threaded diagonally through the nerve stimulating

Another purpose of this invention is to provide a novel hands-free nerve stimulating ball where the string or strap is threaded horizontally through the nerve stimulating ball.

Yet another purpose of this invention is to provide a novel hands-free nerve stimulating ball where an adjustable screw means connects the nerve stimulating ball so that the distance between the at: least two nerve stimulating balls can be easily adjusted.

Therefore, in one aspect this invention comprises a nerve stimulating ball apparatus, comprising:
(a) at least two substantially round balls, and wherein said at least two round balls are connected each with the other to form a substantially peanut shaped ball having at least two peak areas, and at least one valley area;
(b) at least one opening between said at least two substantially round balls, such that at least one strap having a first end and a second end is threaded from a first end of a first ball to a second end in a second ball; and
(c) wherein at least one securing means at said first end of said at least one strap is secured to said second end of said at least one strap so as to slidably secure said peanut shaped ball to said strap.

In another aspect this invention comprises a method of using a spine nerves stimulating ball system, comprising, the steps of:
(a) taking at least two substantially round balls, and wherein said at least two round balls are connected each with the other to form a substantially peanut shaped ball having at least two peak areas, and at least one valley area;
(b) securing at least one adjustable securing means to each of said at least two substantially round balls, such that each of said at least two substantially round balls are free to rotate about its peak areas;
(c) positioning said at least two substantially round balls along a spine of a user such that a first ball is on a right side of said spine, and said second ball is on a left side of said spine; and
(d) rotatably moving said at least two substantially round balls along said spine so as to provide stimulation of thoracic nerves of said user along said spine.

### BRIEF DESCRIPTION OF THE DRAWINGS

FIG. 1 is a perspective view of an inventive peanut or dumbbell shaped nerve stimulating ball with strap inserted through the peanut or dumbbell shaped ball diagonally, according to an embodiment of the invention.

FIG. 2 is a perspective view of an inventive peanut or dumbbell shaped nerve stimulating ball with strap inserted through the peanut or dumbbell shaped ball horizontally, according to an embodiment of the invention.

FIG. 3 is an enlarged view of an inventive peanut or dumbbell shaped nerve stimulating ball configured to receive a strap diagonally.

FIG. 4 is an enlarged view of an inventive peanut or dumbbell shaped nerve stimulating ball configured to receive the strap horizontally.

FIG. 5 is a side view of an inventive peanut or dumbbell shaped nerve stimulating ball that is designed to receive a strap horizontally.

FIG. 6 illustrates a human reflexology chart of a back of a user with major pressure points.

FIG. 7 illustrates the hands-free nerve stimulating ball system invention in use on the back of a user.

FIG. 8 is a front isometric view of a car seat with the hands-free nerve stimulating ball system invention strapped around the car seat.

FIG. 9 is a back isometric view of a car seat with the hands-free nerve stimulating ball system invention strapped around the car seat.

FIG. 10 illustrates the hands-free nerve stimulating ball system invention in use while a user is sitting on a chair.

FIG. 11 illustrates the hands-free nerve stimulating ball system invention in use while a user is driving a vehicle.

FIG. 12 is a perspective view of an inventive nerve stimulating ball system with a string secured to the ball, according to an embodiment of the invention.

FIG. 13 is a perspective view of an inventive nerve stimulating ball system with a string looped and secured to the ball, according to an embodiment of the invention.

### DETAILED DESCRIPTION

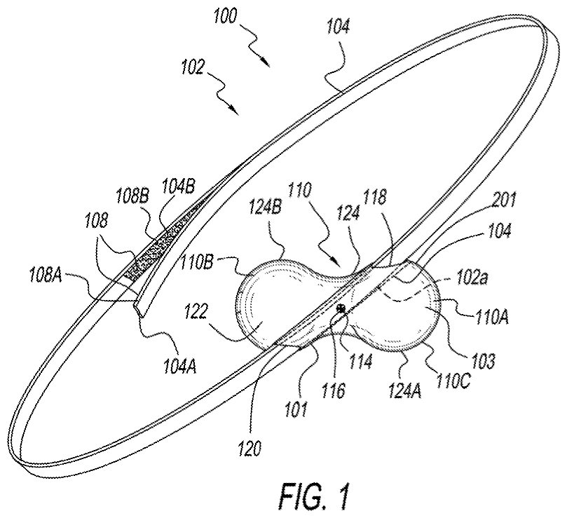

The inventive hands-free nerve stimulating ball apparatus, and a method of using same will now be discussed with reference to FIGS. 1 through 13. Although the scope of the present invention is much broader than any particular embodiment, a detailed description of the preferred embodiment follows together with drawings. These drawings are for illustration purposes only and are not drawn to scale. Like numbers represent like features and components in the drawings.

Furthermore, disclosed herein are detailed descriptions of several specific embodiments of the hands-free nerve stimulating ball, methods, and package assemblies of the present inventions. It is understood that the disclosed embodiments are merely examples of the way in which certain aspects of the invention can be implemented and do not represent and exhaustive list of all the ways the invention may be embodied. Indeed, it will be understood that the systems, devices, methods, and package assemblies described herein may be embodied in various, and alternative forms. The figures are not necessarily to scale, and some features may be exaggerated or minimized to show details of particular components. Well-known components, materials or methods are not necessarily described in great detail in order to avoid obscuring the present disclosure. Any specific structural and functional details disclosed herein are not to be interpreted as limiting, but merely as a basis for the claims, and as a representative basis for teaching one skilled in the art to variously employ the invention.

FIG. 1 is a perspective view of an inventive peanut or dumbbell shaped nerve stimulating ball system 100, with at least one string or strap 104, inserted through the peanut or dumbbell shaped ball 110, diagonally, according to an embodiment of the invention. As shown in FIG. 1, a nerve stimulating ball, and strap system 100, comprising of at least two balls 110A, 110B, which are joined to form at least one peanut or dumbbell shaped balls 110, and which balls 110A, 110B, are smoothly merged together to form a peanut shaped peanut or dumbbell shaped ball surface or shape 110C. FIG. 1, further shows that a diagonal strap loading ball system 102, and strap 104, can be secured with at least one securing means 108, such as, for example, a hook and loop system 108, wherein at least one or first securing means 108A, situated at one or first end 104A, of the strap 104, is secured to at least one or second securing means 108B, situated at another or second end 104B, of the strap 104, so as to secure each end 104A, 104B, each to the other. As shown in FIG. 1, the peanut or dumbbell shaped ball 110, has at least one strap entryway or opening or through-hole 120, which opening is preferably located on a first side or a front side 101, of the peanut or dumbbell shaped ball 110, and a corresponding strap entryway out or exit 118, located on a second or back or rear side 201, of the peanut or dumbbell shaped ball 110. For some applications, on the upper side 103, or lower side 103, or bottom side 103, or top side 103, of the peanut or dumbbell shaped ball 110, one could also have at least one opening or hole 116, so that the strap 104, could be secured to the peanut or dumbbell shaped ball 110, by means of at least one strap screw or strap securing device 114. Also identified in FIG. 1, on the diagonal strap loading ball system 102, is at least one diagonal strap passageway or hole or opening 102a. For most applications it is preferred that the strap 104, is inserted into the passageway 102a, of the peanut or dumbbell shaped ball 110, so that there will be no need for the user 140, to secure the strap 104, to the peanut or dumbbell shaped ball 110. As shown in FIG. 1, the two conjoined peanut or dumbbell shaped balls 110, protrude in an outwardly direction, as shown by reference or peak “124A” and “124B”, so as to form an undulation or a channel or valley 124. For some applications it. is preferred that the outer or upper surface 122, of the peanut or dumbbell shaped ball 110, is substantially smooth 122, or has a smooth finish 122.

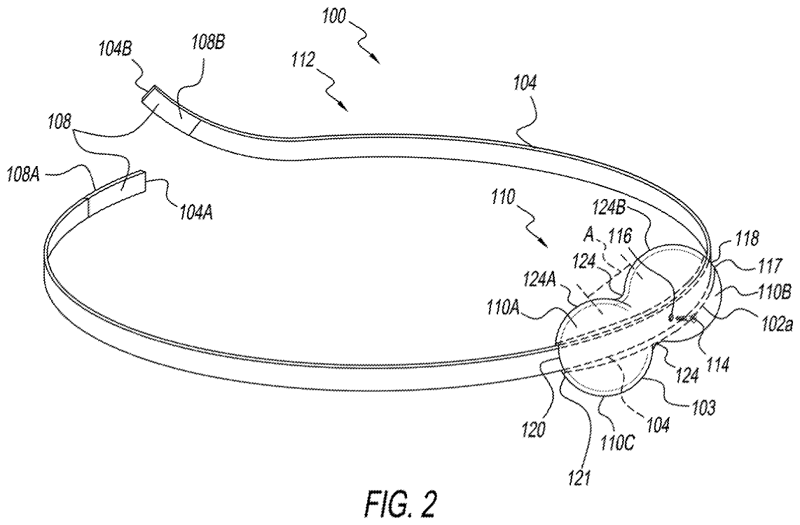

FIG. 2 is a perspective view of an inventive peanut or dumbbell shaped nerve stimulating ball system 100, with at least one string or strap 104, inserted through the peanut or dumbbell shaped ball 110, horizontally, according to an embodiment of the invention. As shown in FIG. 2, a nerve stimulating ball, and strap system 100. comprising of at least two balls 110A, 110B, which are joined to form at least one peanut or dumbbell shaped balls 110, and which balls 110A, 110B, are smoothly merged together to form a peanut shaped peanut or dumbbell shaped ball surface or shape 110C. FIG. 2, further shows that a horizontal strap loading ball system 112, and strap 104, can be secured with at least one securing means 108, such as, for example, a hook and loop system 108, wherein at least one or first securing means 108A, situated at one or first end 104A, of the strap 104, is secured to at least one or second securing means 108B, situated at another or second end 1048, of the strap 104, so as to secure each end 104A, 104B, each to the other. As shown in FIG. 2, the peanut or dumbbell shaped ball 110, has at least one strap entryway or opening, or through-hole 120, which opening 120, is preferably located on a first side or a left side 121, of the peanut or dumbbell shaped ball 110, and a corresponding strap entryway out or exit 118, located on a second or back or right side 117, of the peanut or dumbbell shaped ball 110. For some applications, on the front side 103, or back side 103, or top side 103, or bottom side 103, of the peanut or dumbbell shaped. ball 110, one could have at least one opening or hole 116, so that the strap 104, could be secured to the peanut or dumbbell shaped ball 110, by means of at least one securing means 114, such as, fix example, a strap screw 114. Also identified in FIG. 2, on the horizontal strap loading ball system 112, is the at least one horizontal strap passageway or hole or opening 102a. As shown in FIG. 2, the two conjoined peanut or dumbbell shaped balls 110, protrude in an outwardly direction, as shown by reference or peak “124A” and “124B”, so as to form an undulation or a channel or valley 124, and where the distance between the two peaks 124A, and 124B, is “A”.

For some applications the nerve stimulating balls 110A, 110B, could be about 1.68 inches in diameter, or for example, almost the size of a regular golf ball, however, the size of the nerve stimulating balls 110A, 110B, could vary based on a user's 140, body type, and needs. The nerve stimulating balls 110A, 110B, could be made of, for example, rubber-like material, plastic, wood, metal, stainless steel, and combinations thereof, to name a few. For some applications it is preferred that a bouncing rubber ball 110, whose surface pressure density will be similar to that of a baseball be used.

For some applications the strap 104, could be about 25 inches long on each side with approximately 5 inches to about 10 inches of the tip mounted with a hook 108A, and loop 108B, system. As shown in FIG. 1, to load the strap 104, into the diagonal strap loading ball system 102, the user 140, will first insert, for example, the strap end 104A, of the strap 104, into the strap entryway 120, as shown on the front side 101, of the peanut or dumbbell shaped ball 110. The strap 104, would then be pushed in, and the strap 104, would then slide through the diagonal slide passageway 102a, of the peanut or dumbbell shaped ball 110. The strap 104, would then come out in the rear side 201, of the peanut or dumbbell shaped shape ball 110, by way of the diagonal strap passageway out 118. The same method would be used to insert the strap 104, into the peanut or dumbbell shaped ball 110, having the horizontal strap loading configuration or system 112 as shown in FIG. 2. As shown in FIG. 1 and FIG. 2, after inserting the strap 104, through the passage way in 120, and passageway out 118, the user 140, can further secure the ball 110, in place, on the strap 104, by means of the strap screw 114. The strap screw 114, could be a regular screw 114, such as, for example, a sheet metal or a wood screw 114. The user 140, could secure the peanut or dumbbell shaped ball 110, to the strap 104, by screwing the screw 114, into the ball screw opening or hole 116, on the top surface 103, of the peanut or dumbbell shaped ball 110. The strap screw 114, should have a long enough, and a sharp enough tip 114A, so as to penetrate at least a portion of the strap 104, so as to stop the peanut or dumbbell shaped ball 110, from moving from one side to another side along the strap 104. Once the peanut or dumbbell shaped ball 110, has been secured to a portion of the strap 104, then each end 104A, 104B, of the strap 104, could be secured each to the other, using at least one securing means 108, such as, for example, a hook 108A and loop 108B, system.

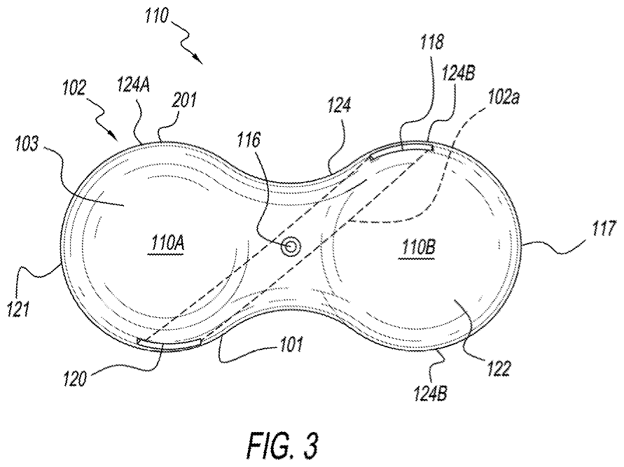

FIG. 3 is an enlarged view of an inventive peanut or dumbbell shaped nerve stimulating ball 100, configured to receive a strap 104, diagonally 102. As one can see in FIG. 3, that the strap passageway 102a, is along a diagonal path between the ball 110A, and ball 110B, so that the strap 104, could enter the peanut or dumbbell shaped ball 110, either from entryway 118, or entryway 120. It is preferred that the at least one opening or hole or blind hole 116, is substantially in a central location, such as, along the valley or lower area 124, so as to secure the strap 104, to the peanut or dumbbell shaped ball 110, and to prevent the sliding movement of either the peanut or dumbbell shaped ball 110, along the strap 104, or the sliding movement of the strap 104, along the passageway 102a, of the peanut or dumbbell shaped ball 110. It should he appreciated that FIG. 3, shows either the top surface 103, of the peanut or dumbbell shaped ball 110, or the bottom surface 103, of the peanut or dumbbell shaped ball 110, as both the top surface 103, and the bottom surface 103, are a mirror image of each other. For some applications it is preferred that the outer or upper surface 122, of the peanut or dumbbell shaped ball 110, is substantially smooth 122, or has a smooth finish 122.

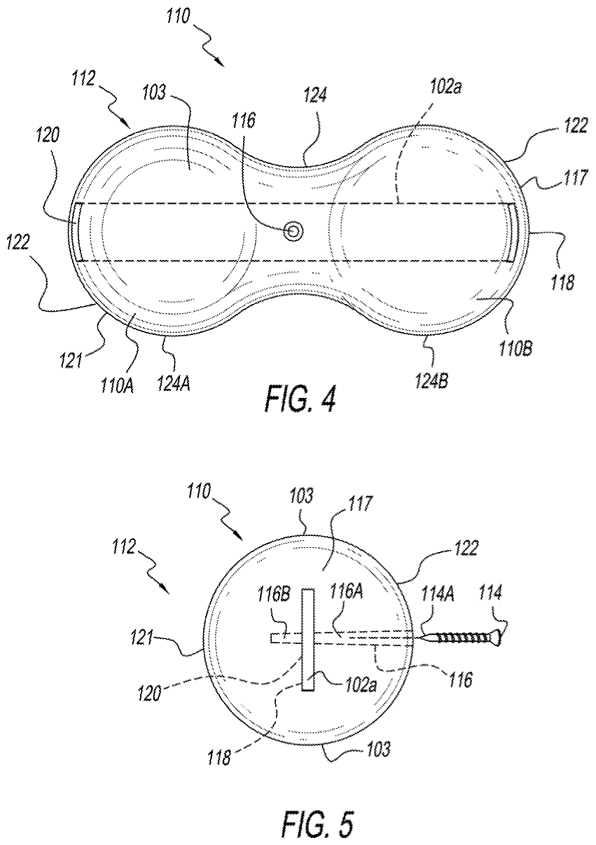

FIG. 4 is an enlarged view of an inventive peanut or dumbbell shaped nerve stimulating ball 100, configured to receive a strap 104, horizontally 112. As one can see in FIG. 4, that the strap passageway 102a, is along a horizontal path between the ball 110A, and ball 110B, so that the strap 104, could enter the peanut or dumbbell shaped ball 110, either from entryway 118, or entryway 120. It is preferred that the opening or hole or blind hole 116, is substantially in a central location, such as, along the valley or lower area 124, so as to secure the strap 104, to the peanut or dumbbell shaped ball 110, and to prevent the sliding movement of either the peanut or dumbbell shaped ball 110, along the strap 104, or the sliding movement of the strap 104, along the passageway 102a, of the peanut or dumbbell shaped ball 110. It should be appreciated that FIG. 4, shows either the top surface 103, of the peanut or dumbbell shaped ball 110, or the bottom surface 103, of the peanut or dumbbell shaped ball 110, as both the top surface 103, and the bottom surface 103, area minor image of each other. For some applications it is preferred that the outer or upper surface 122, of the peanut or dumbbell shaped ball 110, is substantially smooth 122, or has a smooth finish 122,

FIG. 5, is a side view of an inventive peanut or dumbbell shaped nerve stimulating ball 110, that is designed to receive a strap horizontally 112, along at least one opening or strap passageway 102a. As stated earlier that the top surface 103, and the bottom surface 103, are a minor image of each other, similarly, the right side 117, and the left side 121, of the peanut or dumbbell shaped ball 110, are a mirror image of each other. The opening of the strap passageway 118, 120, is shown as a rectangular opening 118, 120, however, the opening 118, 120, can be of any shape, such as, rectangular, 118, 120, square, 118, 120, triangular, 118, 120, round, 118, 120, oval, 118, 120, pentagonal, 118, 120, multisided, 118, 120, and combinations thereof, to name a few. it should be appreciated that the opening 116, for the strap screw 114, having a sharp pointed tip 114A, could comprise of a through hole or opening 116A, so that the strap screw 114, secures the strap 104, to the peanut or dumbbell shaped ball 110. For some applications the opening 116, could extend past the strap passageway 102a, so as to create a blind hole 116B, so that the sharp tip 114A, of the strap screw 114, could pass-through the strap 104, and be embedded into the blind hole or opening 116B, and this would prevent any sliding movement of the string or strap 104.

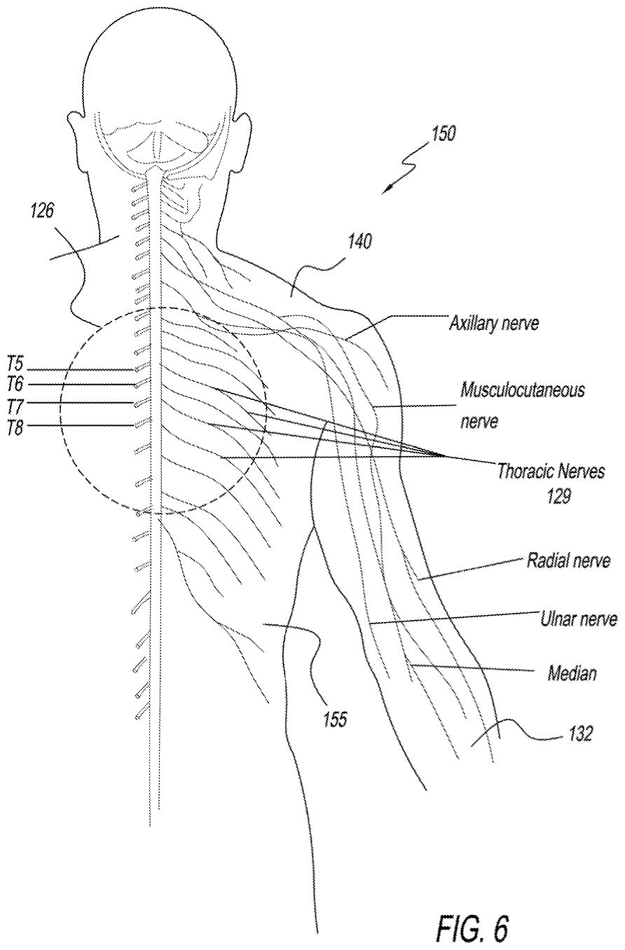

FIG. 6 illustrates a human reflexology chart 150, of a back 155, of a user 140, with major pressure points. The human reflexology chart 150, is on the vertebral column, where the pressure point area of interest 126, or thoracic nerve region 126, of interest is shown or highlighted 126. Reflexology, also known as zone therapy, is the application of pressure to areas, or zones, of the body, such as, for example, the feet, the hands, the areas of the back 155, etc., of a user 140. The zone therapy is based on the principle that there are small, and specific areas of nerve endings in the hands, feet, and back 155, of a user 140, which directly corresponds to specific muscle groups, or organs, of the body, and glands, and can trigger a reflex on those organs, of a user 140. As shown in FIG. 6, which is focusing on, for example, the area of the thoracic nerve 129, T6, that is mapped with the digestive organ, and all the body parts associated with it, like, for example, the stomach. Reflexology therapy helps trigger a reflex response from the corresponding organs, when a gentle but firm pressure is applied to the specific reflex zones on the body of the user 140, namely, the back 155. The hands-free nerve stimulating ball system 100, has been invented to assist with effective pressure application in situations where a user 140, is preferably sitting or driving, and wherein the user 140, may not be able to administer the pressure in the area of, for example, the thoracic nerves 129, located on the back 155, along T5, T6, T7, T8, using his or her own hands, or with the help of a practitioner.

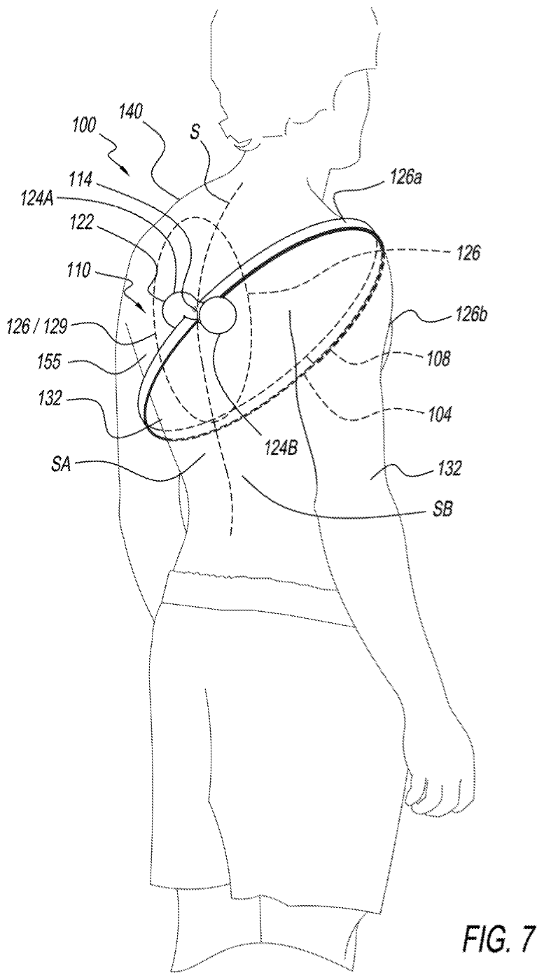

FIG. 7 illustrates the hands-free nerve stimulating ball system invention 100, in use on the back 155, of a user 140. As shown in FIG. 7, the peanut or dumbbell shaped shape ball 110, which is already securely attached to the strap 104, and secured by means of a securing means 114, such as, a screw 114, is wrapped around a user 140. The user 140, securely places the peanut or dumbbell shaped balls 110, of the nerve stimulating system 100, to the desired pressure point, such as, for example, pressure point area 126, 129, of the hack 155, by means of the strap 104. For the purposes of illustration the user 140, could securely place the peanut or dumbbell shaped ball 110, to the back 155, by pulling on one end 104A, of the strap 104, over the right shoulder 126a, and then pulling the other or second end 104B, of the strap 104, under the arm 132, of the user 140, and then securing both the strap ends or edges 104A, 104B, together at the front chest area 126b, by means of a securing system 108, such as, for example, the hook 108A, and loop 108B, system. The more the user 140, tightens the securing means 108, together the more the strap 104, will secure the peanut or dumbbell shaped ball 110, to the back 155, of the user 140. Once the strap 104, having the peanut or dumbbell shaped ball 110, have been secured around the user's 140, body, the user 140, now has both hands free, and thus the user 140, can now perform other activities, such as, sitting, driving, standing, or even lying down on the back 155, as during normal user's 140, movement, the smooth surface 122, or the peak surface 124A, 124B, of the peanut or dumbbell shaped ball 110, would rub or massage the thoracic nerve region 126, and would stimulate the thoracic nerves 129, on the back 155, of the user 140. Thus, is should be appreciated that basically, it is the peak area or surface 124A, 124B, of the peanut or dumbbell shaped ball 110, that comes in direct contact with the back 155, and the thoracic nerve region 126, 129, of the user 140, and it is that contact that stimulates and provides the massage to the thoracic nerve region 126, 129 it should be appreciated that the spine S, has a first side or left side spine area SA, and a second side or right side spine area SB, so that the peak area or surface 124A, would be used to massage or stimulate the first side or left side spine area SA, while the peak area or surface 124B, would be used to massage or stimulate the second side or right side spine area SB.

It should be noted that means of tightening the strap 104, is not limited to the hook 108A, and loop 108B, system or material. Any method of buckling or tightening or securing of two loose ends 104A, 104B, together could be used to tighten the strap 104, to the body of the user 140. Whatever method is used, the result will be that the nerve stimulating hall 110, will be strapped in place on the back 155, of the user 140. As shown in FIG. 2, the two peanut or dumbbell shaped balls 110, protrudes outwardly, as shown by reference “124A” and “124B” and forms a connecting channel 124. The shape of the peanut or dumbbell shaped ball 110, is so designed to protect the spine area “S” of the user 140, so that pressure is applied to the thoracic nerve endings 129, and not the spine “S” when the peanut or dumbbell shaped ball 110, is applied to the back 155, of the user 140, and is in use, as illustrated in FIG. 7. Thus, for example, peak areas 124A, would be, for example, on the first or left side SA, of the spine S, of the user 140, while the peak area 124B, would be on the second or right side SB, of the spine S, of the user 140.

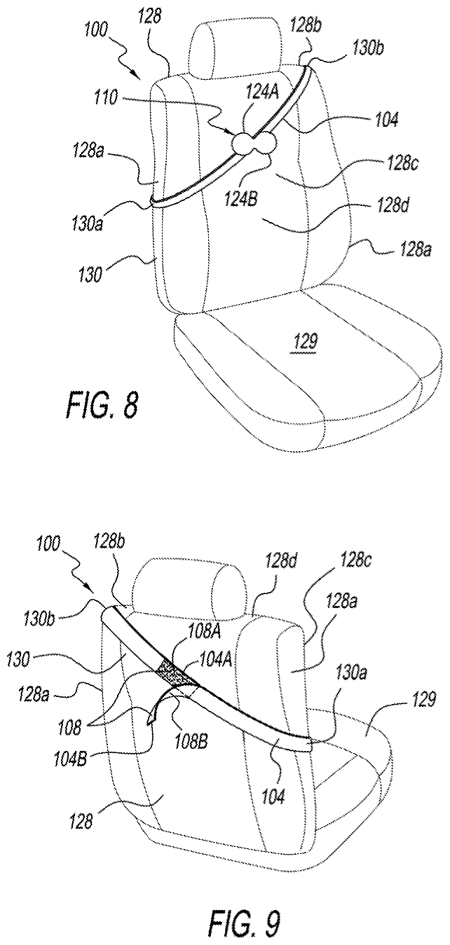

FIG. 8 is a front isometric view of a car seat 128, with the hands-free nerve stimulating ball system invention 100, strapped around the car seat 128, while FIG. 9 is a back isometric view of a car seat 128, with the hands-free nerve stimulating ball system invention 100, strapped around the car seat 128. The car seat 128, has a side 128a, a seat top 128h, a seat front side 128c, a seat back rest 128d, a seat back-side 130, and a sitting area 129. The hands-free nerve stimulating ball system 100, is shown attached to and around a car seat 128, instead of being worn by a user 140. This method of use of the hands-free nerve stimulating ball system invention 100, is beneficial for a situation, such as, for example, when the user 140, wants the invention to work while the user 140, is doing another task, such as, driving. The nerve stimulating ball system 100, is placed around the car seat 128, such that the peanut or dumbbell shaped balls 110, are secured, and strapped to the front side 128c, of the back rest 128d, around a location which will come in contact with the back 155, of the user 140, especially, the thoracic nerve region 126, of the user 140. The peanut or dumbbell shaped ball 110, can be attached to the strap 104, and the strap 104, can also be secured around the car seat 128, in a similar manner as described in FIG. 7, wherein the user 140, swings one end of the strap 104, over the surface designated as a chair top wrap 130b, around the area designated as chair or seat top 128b, and the other end 104A, 104B, around the area designated as chair or scat side wrap 130a, around the seat side 128a, of the chair 128, and around to the back 130, of the chair 128, as more dearly shown in FIG. 9.

The nerve stimulating ball 100, with peanut or dumbbell shaped shape 110, are positioned or placed on the front side 128c, of the chair or seat 128, with more of the peanut or dumbbell shaped ball 110, facing towards the user 140, who will sit on the seating area 129, of the car seat or chair 128. The strap screw 114, located on the top side 103, of the peanut or dumbbell shaped ball 110, can be positioned close to the car seat or chair 128, such that they are not visible to the user 140. FIG. 9 further illustrates the manner in which the peanut or dumbbell shaped ball 110, stay in place, and do not fall down due to gravity. The user 140, can tighten the strap 104, in the back 130, of the chair 128, by means of the loop 108A, and hook 108B, system, which are at the tip end 104A, 104B, of each strap 104. This allows the peanut or dumbbell shaped ball 110, to remain at the proper level of the back 155, thoracic region 126, 129, when the user 140, sits in the chair or car seat 128. This method of using the nerve stimulating ball system 100, can serve several purposes, such as, the user 140, can have it secured to user's 140, favorite chair 128, or car seat 128. Similarly, the user 140, can have one secured to user's 140, car seat 128, one secured to the user's 140, office chair 128, one secured to the user's 140, chair 128, at home, etc. Having the inventive nerve stimulating ball 100, with peanut or dumbbell shaped shape 110, at multiple locations will also help the user 140, to save time, instead of manually strapping the inventive nerve stimulating ball 100, with peanut or dumbbell shaped shape 110, to a user's 140, body.

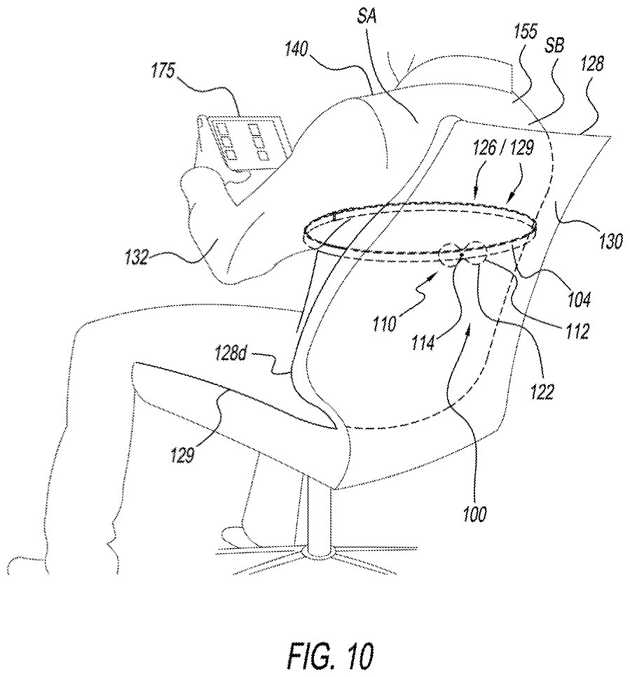

FIG. 10 illustrates the hands-free nerve stimulating ball system 100, invention in use while a user 140, is sitting on a chair 128. After the user 140, straps the hands-free nerve stimulating ball system 100, invention, as shown, and described in FIG. 7, the user 140, can now sit down on a chair 128, having a back rest 128d, such that the peanut or dumbbell shaped ball 110, are positioned between the back 155, of the user 140, and the back rest 128d, around the thoracic nerve region 126, 129, and then the user 140, can press against the back rest 128d, using the user's 140, back 155, such that the peanut or dumbbell shaped ball 110, provide a massaging movement or massaging therapy to the thoracic nerve region 126, 129, on the back 155, of the user 140, As shown in FIG. 10, the user 140, could be sitting on a. chair 128, in an office setting, or the user 140, could be at home, or some other location. The hands-free nerve stimulating ball 100, are securely strapped on to the user's 140, body. It should be noted that the user 140, is using the hands-free nerve stimulating ball 100, with a horizontal strap loading 112, peanut or dumbbell shaped ball 110. However, either horizontal peanut or dumbbell shaped ball system 112, or diagonal peanut or dumbbell shaped ball system 102, configuration can be used.

During use or operation the strap 104, is wrapped under both arms 132, of the user 140, and secured in the front with the hook 108A, and loop 108B, system, at the strap ends 104A, 104B. The strap 104, is wrapped under both arms 132, around the chest area 126b, of the user 140, and secured in the front with the hook 108A, and loop 108B, system, at the strap ends 104A, 104B, such that the user's 140, arms 132, are free to do another task, such as, operate a device 175, such as, for example, an electronic device 175.

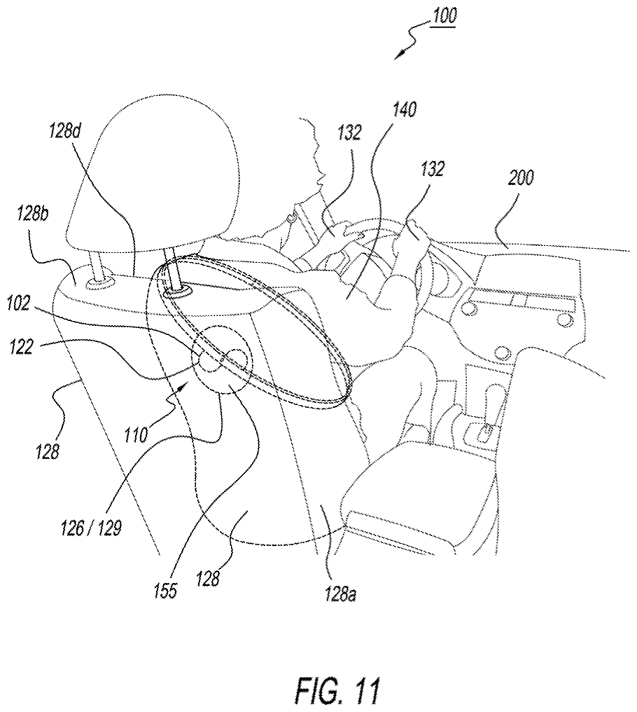

FIG. 11 illustrates the hands-free nerve stimulating ball system 100, invention in use while a user 140, is driving a vehicle 200. After the user 140, straps the hands-free nerve stimulating ball system 100, invention, as shown, and described in FIG. 7, the user 140, can now sit down on a car seat 128, having a back rest 128d, such that the peanut or dumbbell shaped ball 110, are positioned between the back 155, of the user 140, and the back rest 128d, around the thoracic nerve region 126, 129, and then the user 140. can press against the back rest 128d, using the user's 140, back 155, such that the peanut or dumbbell shaped ball 110, provide a massaging movement or massaging therapy to the thoracic nerve region 126, 129, on the back 155, of the user 140. As shown in FIG. 11, the user 140, is sitting on a car seat 128, and driving a car 200, or a vehicle 200. The hands-free nerve stimulating ball 100, are securely strapped on to the user's 140, body. It should be noted that the user 140, is using the hands-free nerve stimulating ball 100, with a diagonal strap loading system 102, peanut or dumbbell shaped ball 110. However, either diagonal peanut or dumbbell shaped ball system 102, or horizontal peanut or dumbbell shaped ball system 112, configuration can be used. The strap 104, is wrapped under both arms 132, around the chest area 126b, of the user 140, and secured in the front with the hook 108A, and loop 108B, system, at the strap ends 104A, 104B, such that the user's 140, arms 132, are free to do another task, such as, for example, drive a vehicle 200.

It should be noted that either the user 140, can simply sit in a strap loaded chair 128, or car seat 128, as shown in FIGS. 8-11, or the user 140, can put or wear the strap 104, on the user's 140, body horizontally 112, as shown in FIG. 10, or diagonally 102, as shown in FIG. 11. All method of using the hands-free nerve stimulating ball system 100, invention will yield the same desire result, i.e., hands-free nerve stimulating massage. Whichever orientation the strap 104, is placed on the body, the peanut or dumbbell shaped ball 110, will always be along a horizontal plane or axis.

In view of the above, it must be noted that simply wearing the nerve stimulating ball 100, will not in itself facilitate the massage pressure on the user's 140, thoracic nerve region 126, 129. It should be noted that in FIG. 10, and FIG. 11, when the strap 104, is tightened around the chest of the user 140, using a securing means 108, such as, hook 108A, loop 108B, system, the peanut or dumbbell shaped ball 110, whether strung horizontally 112, as shown in FIG. 10, or strung diagonally 102, as shown in FIG. 11, the peanut or dumbbell shaped ball 110, tend to stay in place almost horizontal to the vertical spine S, in and around the thoracic nerve area 126, of the back 155, of the user 140, around the area of the thoracic nerve 129, as shown in FIG. 6. To achieve the useful result of the invention, the user 140, must press against another back rest surface 128d, which is hard or semi-hard, or can push back the pressure that is being placed or applied by the back 155, of the user 140. The pressure, and force, applied to the peanut or dumbbell shaped ball 110, located between the back 155, of the user 140, and the back rest surface 128d, will stimulate the thoracic nerves 129, to achieve desire results.

The back rest surface 128d, that the user 140, can press against depends on what the user 140, is doing, for example, as shown in FIG. 8, and FIG. 9, the back rest surface 128d, is a back rest 128d, of the car seat 128, while FIG. 10, shows that the back rest. surface 128d, to be an office chair 128, and FIG. 11, shows the back rest surface 128d, to be a driver 140, or a passenger 140, seated in a motor vehicle 200. Thus, a user 140, has two different ways to use this hands-free nerve stimulating ball system 100, invention, one way is to simply sit in a seat or a chair 128, that already has the hands-free nerve stimulating ball system 100, invention secured thereto, and apply the desired pressure onto the thoracic nerve region 126, or to wear the hands-free nerve stimulating ball system 100, invention, and then press the peanut or dumbbell shaped ball 110, against a back rest 128d, in order to apply the desired pressure against the thoracic nerve region 126. Thus, either way, a user 140, using the hands-free nerve stimulating ball system 100, invention, and applying pressure against the back 155, around the thoracic nerve region 126, will help facilitate the needed pressure, and massage, to achieve the desire digestive results, such as, burping, and other digestive benefits, as stated. earlier. Furthermore, this invention can be used immediately after a meal. To assist with, for example, digestion, a user 140, can use the hands-free nerve stimulating ball system 100, invention. The hands-free nerve stimulating ball system 100, invention is portable enough to fit in a bag, or a pouch, and can either be taken, or be strapped to a car seat 128, or a chair 128, or a similar such structure 128.

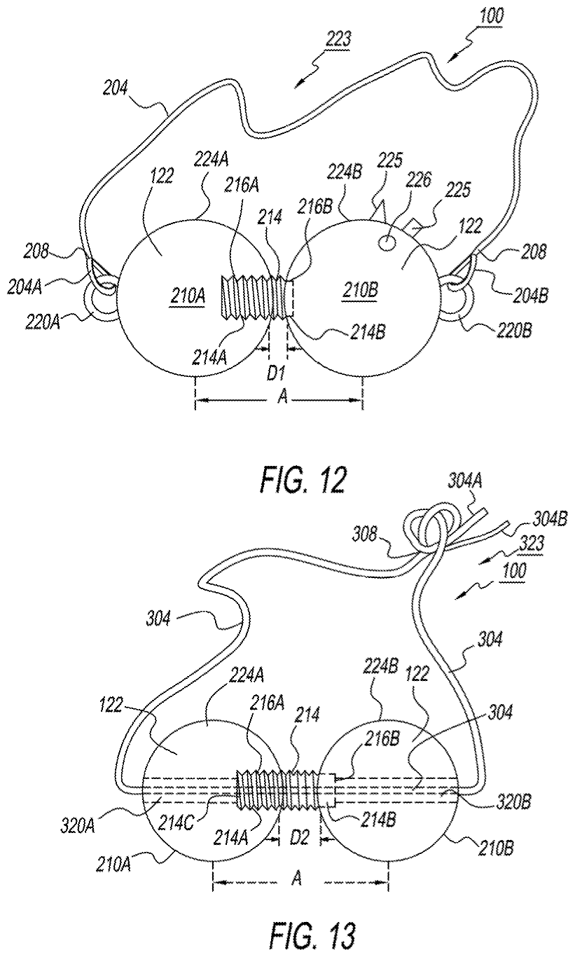

FIG. 12 is a perspective view of art inventive nerve stimulating ball system 100, with a strap or string 204, secured to the ball 210A, 210B, according to an embodiment of the invention 223. The inventive nerve stimulating ball system 223, comprises of at least two substantially round ball 210A, 210B, having at least one opening or hole 216A, 216B, respectively. Each of the round ball 210A, 210B, has at least one catch 220A, 220B, respectively secured thereto. At least one string or strap 204, having a first end 204A, and a second end 204B, is secured to the ball 210A, 210B, such that, for example, first end 204A, is secured to the catch 220A, while the second end 204B, is secured to the catch 220B. At least one connector 214, is used to connect the ball 210A, to the ball 210B, using the blind hole 216A, 216B, respectively. It is preferred that the at least one connector 214, is a screw connector 214, such that by rotating either ball 210A, or 210B, the distance “D1” between ball 210A, and ball 210B, can easily be adjusted, so that the user 140, can adjust the peak location 224A, 224B, by a distance D1, so as to make sure that the peak location 224A, 224B, are reaching and properly stimulating the thoracic nerve region 126, 129, along the spine S, especially, on the left side SA, and the right side SB, of the spine S. It should be appreciated that each person or user 140, has a different height, weight, chest area, and thus a differing thoracic nerve region or area 126, 129, and thus having the ability to adjust the distance D1, between the two peaks 224A, 224B, allows the user 140, to customize the inventive nerve stimulating ball system 223, for their particular needs, as it is basically, the peak area 224A, 224B, that comes in direct contact with the back 155, and the thoracic nerve region 126, 129, of the user 140, and it is that contact that stimulates and provides the massage to the thoracic nerve region 126, 129. For some applications one could use at least one securing means 208, to secure the string or strap ends 204A, 2048, to the corresponding eyelet 220A, 220B. For some applications the at least one securing means 208, could be selected from a group comprising an adhesive 208, a glue 208, a weld 208, a knot 208, a hook and loop system 208, and combinations thereof, to name a few. For some applications the outer or upper smooth surface 122, of the ball 110, 210A, 210B, could have one or more protrusion radiating outwardly 225, or inwardly radiating undulation or blind hole 226, such as, for example, a golf ball like dimple 226, or an inverse egg shaped feature 226, or an inverse structure 226, so that the features 225, and/or 226, on the exterior or outer surface 122, act like mini-massaging fingers 225, 226. The catch 220A, 2208, could be, for example, an eyelet type of a catch 220A, 220B, through which the string or strap 204, could be threaded through. For some applications one or first end 214A, of the connector or screw 214, could be securely fixed to one ball 210A, 210B, such as, for example, into the opening or blind hole 216A, of ball 210A, while the other or second end 214B, could be rotatably secured inside the blind hole or opening 216B, of ball 210B, so that by the rotation of either ball 210A, or ball 210B, the distance D1, between ball 210A, and ball 210B, could be easily adjusted, to a different position, such as, for example, distance D2, (FIG. 13), which would also result in the adjustment of the distance A, between the peak region 224A, 2248, or the outer surface 122, between ball 110, 210A, 210B, such as, connected via at least one connector 214. Thus, a user or consumer 140, has the option of adjusting or bringing the spine nerves stimulating balls 210A, 210B, either closer together or separating them further apart, in order to provide the appropriate stimulation to the thoracic nerve region 126, 129.

FIG. 13 is a perspective view of an inventive nerve stimulating ball system 100, with a strap or string 304, looped, and secured to the nerve stimulating balls 210A, 210B, according to an embodiment of the invention 323. The inventive nerve stimulating ball system 323, of FIG. 13, is similar to the inventive nerve stimulating ball system 223, shown in FIG. 12, except that the distance D2, between the peak areas 224A, 224B, is different, a different strap or string 304, having ends 304A, 304B, is being used. The string or strap 304, could have at least one securing means 308, to secure strap ends 304A, 304B, each to the other, such as, for example, a knot 308, an adhesive 308, a glue 308, a weld 308, a hook and loop system 308, and combinations thereof, to name a few. For some applications one or first end 214A, of the screw or connector 214, could be securely fixed to one ball 210A, 210B, such as, for example, into the opening or blind hole 216A, of ball 210A, while the other or second end 214B, could be rotatably secured inside the blind hole or opening 216B, of ball 210B, so that by the rotation of either ball 210A, or ball 210B, the distance D1, between ball 210A, and ball 210B, could be easily adjusted, to a different position, such as, for example, distance D2, which would also result in the adjustment of the distance A, between the peak region 224A, 224B. Thus, a user or consumer 140, has the option of adjusting or bringing the spine nerves stimulating balls 210A, 210B, either closer together or separating them further apart, in order to provide the appropriate stimulation to the thoracic nerve region 126, 129. For some applications, the connector or screw 214, could have a hollow center 214C, or having an opening or passageway 214C, for the passage of the string or strap 304. And, so similarly, the spine nerves stimulating balls 210A, 210B, could also have an opening or passageway 320A, 320B, respectively, for the passage of the string or strap 304.

The cross-sectional area for the finger or protrusion 225, can be selected from a group comprising a triangle, a square, a rectangle, a circle, an oval, a polygonal shape, a cylindrical shape, and combinations thereof, to name a few. Similarly, the cross-sectional area for the inwardly radiating undulation or blind hole 226, can be selected from a grout comprising a triangle, a square, a rectangle, a circle, an oval, a polygonal shape, a cylindrical shape, and combinations thereof, to name a few.

The peanut or dumbbell shaped ball 110, or ball 210A, 210B, can be made from a material selected from a group comprising plastic, wood, rubber, metal, brass, copper, stainless steel, glass, quartz, polymeric material, composite material, and combinations thereof, to name a few. Additionally, the peanut or dumbbell shaped ball 110, or ball 210A, 210B, can be made of different radius or diameter, as the back 155, of the spine S, has a curved shape, and so the diameter or the radius of the peanut or dumbbell shaped ball 110, or ball 210A, 210B, can be different is size, as long as each of the peanut or dumbbell shaped ball 110, or ball 210A, 210B, comes in contact with the desired thoracic. nerve region 126, 129, along the spine S, on the back 155, of the user 140, so even if the peanut or dumbbell shaped ball 110, or ball 210A, 210B, are even or uneven, as along as each peak region 124A, 124B, comes in contact with at least a portion of the thoracic nerve region 126, 129, around the spine S.

All structural and functional equivalents to the elements of the various embodiment of the invention described throughout the disclosure that are known or later come to be known to those ordinarily skilled in the art are expressly incorporated herein by reference and intended to be encompassed by the invention.

The above description and drawings are only illustrative of the preferred embodiments which achieve the objectives, features and advantages of the present invention and it is not intended that the present invention be limited thereto. Any modification of the present invention which comes within the spirit and scope of the following claims is considered part of the present invention. Furthermore, to the extent that the terms “include”, “have” or “like” is used in the description or the claims, such terms is intended to be inclusive in manners similar to the term “comprise” as interpreted when employed as a transitional word in the claim.

Thus, the present invention is not limited to the embodiments described, herein and the constituent elements of the invention can be modified in various manners without departing from the spirit and scope of the invention. Various aspects of the invention can also be extracted front any appropriate combination of a plurality of constituent elements disclosed in the embodiments. Some constituent elements may be deleted in all of the constituent elements disclosed in the embodiments. The constituent elements described in different embodiments may be combined arbitrarily.

Still further, while certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions.

It should be further understood that throughout the specification and claims several terms have been used and they take the meanings explicitly associated herein, unless the context clearly dictates otherwise. For example, the phrase “in one embodiment” as used herein does not necessarily refer to the same embodiment, though it may. Additionally, the phrase “in another embodiment” as used herein does not necessarily refer to a different embodiment, although it may. Thus, various embodiments of the invention may be readily combined, without departing from the scope or spirit of the invention.

While the present invention has been particularly described in conjunction with a specific preferred embodiment, it is evident that many alternatives, modifications and variations will be apparent to those skilled in the art in light of the foregoing description. lit is therefore contemplated that the appended claims will embrace any such alternatives, modifications and variations as falling within the true scope and spirit of the present invention.

What is claimed is:

1. A nerve stimulating ball apparatus, comprising:
(a) at least two substantially round balls, and wherein said at least two round balls are connected each with the other to form a substantially peanut shaped ball having at least two peak areas, and at least one valley area;
(b) at least one opening between said at least two substantially round balls, such that at least one strap having a first end and a second end is threaded from a first end of a first ball to a second end in a second ball; and
(c) wherein at least one securing means at said first end of said at least one strap is secured to said second end of said at least one strap so as to slidably secure said peanut shaped ball to said strap.

2. The nerve stimulating ball apparatus of claim 1, wherein at least one screw secures said peanut shaped ball to said at least one strap so as to prevent said slideable movement of said peanut shaped ball along said at least one strap.

3. The nerve stimulating ball apparatus of claim 1, wherein said peanut shaped ball is made from a material selected from a group consisting of plastic, wood, rubber, metal, brass, copper, stainless steel, glass, quartz, polymeric material, composite material, and combinations thereof.

4. The nerve stimulating ball apparatus of claim 1, wherein said peanut shaped ball is securely placed around the thoracic nerve region of a user.

5. The nerve stimulating ball apparatus of claim 1, wherein said peanut shaped ball is securely placed around a chair such that it comes in direct contact with the thoracic nerve region of a user.

6. The nerve stimulating ball apparatus of claim 1, wherein said peanut shaped ball is securely placed around a car seat such that it comes in direct contact with the thoracic nerve region of a user.

7. The nerve stimulating ball apparatus of claim 1, wherein at least one structure is formed on an outer surface of said peanut shaped ball.

8. The nerve stimulating ball apparatus of claim 1, wherein at least one structure is formed on an outer surface of said peanut shaped ball, and wherein said at least one structure is selected from a group consisting of a protrusion, a dimple, an undulation, and combinations thereof.

9. The nerve stimulating ball apparatus of claim 1, wherein at least one protrusion is formed on an outer surface of said peanut shaped ball, and wherein said at least one protrusion has a cross-sectional shape selected from a group consisting of a rectangular shape, a square shape, a triangular shape, a round shape, an oval shape, a pentagonal shape, a multisided shape, and combinations thereof.

10. The nerve stimulating ball apparatus of claim 1, wherein said at least one opening in said peanut shaped ball has a cross-sectional shape selected from a group consisting of a rectangular shape, a square shape, a triangular shape, a round shape, an oval shape, a pentagonal shape, a multisided shape, and combinations thereof.

11. The nerve stimulating ball apparatus of claim 1, wherein said nerve stimulating ball apparatus is worn by a user, such that said at least two substantially round balls, are positioned around a thoracic nerves region along the spine of said user.

12. A method of using a spine nerves stimulating ball. system, comprising the steps of:
(a) taking at least two substantially round balls, and wherein said at least two round balls are connected each with the other to form a substantially peanut shaped ball having at least two peak areas, and at least one valley area;
(b) securing at least one adjustable securing means to each of said at least two substantially round balls, such that each of said at least two substantially round balls are free to rotate about its peak areas;
(c) positioning said at least two substantially round balls along a spine of a user such that a first ball is on a right side of said spine, and said second ball is on a left side of said spine; and
(d) rotatably moving said at least two substantially round balls along said spine so as to provide stimulation of thoracic nerves of said user along said spine.
